(19) United States
(12) Patent Application Publication
(10) Pub. No.: US 2004/0068141 A1
(43) Pub. Date: Apr. 8, 2004

(54) Process for the synthesis of trifluorophenylacetic acids

(76) Inventors: Joseph D. Armstrong, Westfield, NJ (US); Spencer D. Dreher, Metuchen, NJ (US); Norihiro Ikemoto, Edison, NJ (US)

Correspondence Address: Philippe L. Durette, Merck & Co., Inc., RY60-30, Patent Dept., P.O. Box 2000, Rahway, NJ, 07065-0907, US

(21) Appl. No.: 10/679,986

(22) Filed: Oct. 7, 2003

Related U.S. Application Data
(60) Provisional application No. 60/416,790, filed on Oct. 8, 2002.
(60) Provisional application No. 60/416,670, filed on Oct. 7, 2002.

(51) Int. Cl. C07C 51/38

(52) U.S. Cl. 562/479; 562/498

(57) ABSTRACT

The present invention addresses a process for the preparation of 2,4,5-trifluorophenylacetic acid using a Cu(I) salt as a catalyst.

### CROSS-REFERENCE TO RELATED APPLICATIONS

The present invention is related to U.S. provisional application Serial. No. 60/416,670, filed Oct. 8, 2002, the contents of which are hereby incorporated by reference.

### BACKGROUND OF THE INVENTION

The present invention relates to processes for the preparation of trifluorophenylacetic acids that are useful as intermediates in the preparation of inhibitors of the dipeptidyl peptidase-IV (“DP-IV” or “DPP-IV”) enzyme. These drugs are useful to treat diabetes, particularly type 2 diabetes. See, for example WO 97/40832, WO 98/19998, U.S. Pat. No. 5,939,560, Bioorg. Med. Chem. Lett., 6, 1163-1166 (1996); and Bioorg. Med. Chem. Lett., 6, 2745-2748 (1996).

The preparation of phenylacetic acid derivatives from aryl halides with varying substituents has been addressed in, for example: Shi, et al., Tetrahedon, 55, 908-918 (1999); U.S. Pat. No. 6,395,921; Lindley, J., Tetrahedron, 40, 1433-1456 (1984); and Setsune, et al., Chem. Ltrs., 367-370, (1981). These references describe the preparation of [bis-(trifluoromethyl)-phenyl]-acetic acids from halide-bis(trifluoromethyl)-benzenes by either copper catalyzed coupling of malonates or alternatively via allylation and oxidation. However, these methods have not been applied to the production of trifluorophenylacetic acids. Indeed, current processes for the preparation of trifluorophenylacetic acids are not amenable to scale-up and preparation of multi-kilogram quantities. In contrast, the present invention provides an effective method for preparing trifluorophenylacetic acid quickly and efficiently. Furthermore, the present invention permits the inclusion of copper(I) chloride as a catalyst rather than copper(I) bromide or iodide, two commonly used catalysts for such a reaction.

It will be appreciated that 2,4,5-trifluorophenylacetic acid is an important intermediate for a particularly useful class of therapeutic agents. Because of the practical use of 2,4,5-trifluorophenylacetic acid, there is a need for the development of a process for its preparation which is amenable to scale-up, and uses cost-effective and readily available reagents.

The process of this invention is an efficient method to produce the large quantities of 2,4,5-trifluorophenylacetic acid required for large-scale synthesis of various diabetes medicines, particularly those targeting the DP-IV enzyme.

In accordance with one aspect of the present invention, 2,4,5-trifluorphenyl-malonate produced by linking diethylmalonate to 1-bromo-2,4,5-trifluorobenzene is subjected to hydrolysis and decarboxylation to form 2,4,5-trifluorophenylacetic acid. The resulting acid purity can be as high as 99% and yields can be as high as 80%, and the two step procedure allows for rapid, cost efficient and large-scale synthesis of the desired acid.

### SUMMARY OF THE INVENTION

A process for the preparation of a compound of the formula 1:

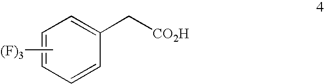

is disclosed comprising: reacting a compound of the formula 3:

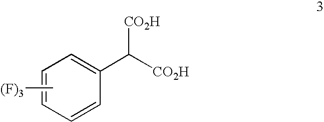

with a decarboxylating agent to produce a compound of formula 4.

### DETAILED DESCRIPTION OF THE INVENTION

The present invention relates to processes for the preparation of trifluorophenylacetic acids. These compounds are intermediates in the synthesis of compounds that are inhibitors of the DP-IV or DPP-IV enzyme, and thus useful in the treatment of diabetes.

The invention is described herein in detail using the terms defined below unless otherwise specified.

Ester refers to a compound that contains the —CO₂— functional group.

The term “alkyl” refers to a monovalent alkane (hydrocarbon) derived radical containing from 1 to 10 carbon atoms unless otherwise defined. It may be straight, branched or cyclic. Preferred alkyl groups include methyl, ethyl, propyl, isopropyl, butyl, t-butyl, cyclopentyl and cyclohexyl. The most common alkyl group used herein is ethyl, represented by “Et”.

Halide and “halo” refer to bromine, chlorine, fluorine and iodine.

Malonate refers to di-esters of the general formula

ROOC—CH₂—COOR

wherein each R represents an alkyl group. Preferred R groups contain to 4 carbon atoms, i.e. forming methyl, ethyl, propyl or butyl malonates.

Suitable deprotonating agents for the process described herein are inorganic and organic bases, for example, alkaline earth metal and alkali metal hydrides, amides, alkoxides, carbonates and bicarbonates, such as sodium hydride, potassium hydride, lithium hydride, sodium amide, sodium methoxide, sodium ethoxide, sodium t-butoxide, potassium tert-butoxide, sodium hydroxide, sodium carbonate, potassium carbonate, lithium carbonate, ammonium carbonate, sodium bicarbonate, potassium bicarbonate, and tertiary amines, such as trimethylamine, triethylamine, tributylamine, diisopropylethylamine (DIPEA), lithium diethylamine, N,N-dimethylaniline, N,N-dimethylbenzylamine, pyridine, N-methylpiperidine, N-methylbenzylamine, N-methylmorpholine, N,N-dimethylaminopyridine (DMAP), 1,4-diazabicyclo[2.2.2]octane (DABCO), 1,5-diazabicyclo[5.4.0]non-5-ene, (DBN), 1,8-diazabicyclo[5.4.0]undec-7-ene (DBU), hexamethyldisilazide, and the like.

The preferred deprotonating agents for use herein include sodium t-butoxide and potassium t-butoxide.

Suitable copper salts are for example copper(I) halides, such as Cu(I)Cl, Cu(I)Br, Cu(I)I and the like. The preferred copper salt is copper (I) chloride.

Acids as used herein refers to acids suitable for decarboxylation, such as HCl, para toluene sulfonic, sulfuric and the like.

In one aspect of the invention a process for the preparation of a compound of the formula 4:

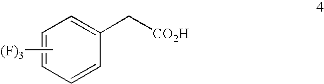

is disclosed comprising:

a) reacting a compound of formula 1:

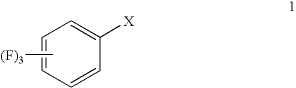

wherein X is a halo group selected from chlorine, bromine and iodine, with a di(C₁₋₄ alkyl)malonate of the formula:

ROOC—CH₂—COOR

wherein each R represents a C₁₋₄ alkyl group, in the presence of a deprotonating agent and a copper (I) salt, to produce a compound of formula 2:

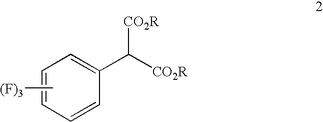

b) reacting compound 2 with a de-esterifying agent to produce a compound of formula 3:

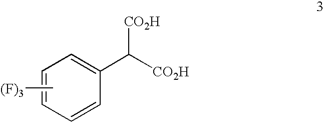

and

c) reacting compound 3 with a decarboxylating agent to produce a compound of formula 4.

An aspect of the invention that is of interest relates to the process disclosed wherein the di(C₁₋₄-alkyl)malonate is diethylmalonate.

Another aspect of the invention that is of interest relates to the process described above wherein the deprotonating agent is sodium tert-butoxide.

Another aspect of the invention that is of interest relates to the process described above wherein the de-esterifying agent is a base, preferably sodium hydroxide, potassium hydroxide or lithium hydroxide.

Another aspect of the invention that is of interest relates to the process described above wherein the Cu(I) salt is selected from CuCl, CuBr and CuI, present in a substoichiometric amount.

Another aspect of the invention that is of particular interest relates to the process described above wherein the reaction is carried out in an aqueous environment.

Another aspect of the invention that is of particular interest relates to the process described above wherein the decarboxylating agent is an acid, preferably hydrochloric acid. More particularly, the amount of hydrochloric acid used is sufficient to adjust the pH to about 0.5 to 1.5.

Another aspect of the invention that is of particular interest relates to the process described above wherein the temperature range is about 45-95° C.

Another aspect of the invention that is of particular interest relates to the process described above wherein the compound of formula 1 is 1-bromo-2,4,5-trifluorobenzene.

Another aspect of the invention that is of particular interest relates to the process described above wherein the compound of formula 4 is 2,4,5-trifluorophenylacetic acid.

In an aspect of the invention that is of particular interest, a process is disclosed wherein the compound of formula 2 is 2,4,5-trifluorophenyl-diethylmalonate.

The general process relates to the preparation of trifluorophenylacetic acids as set forth below.

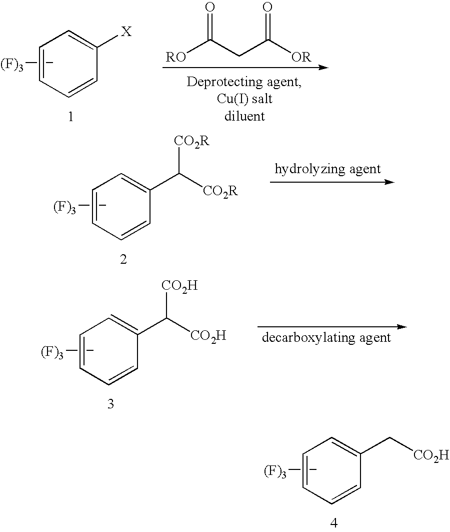

X represents a halide selected from bromine, chlorine and iodine, and each R independently represents a C₁₋₄ alkyl group.

One embodiment of the invention relates to the preparation of 2,4,5-trifluorophenylacetic comprising contacting 1-bromo-2,4,5-trifluorobenzene with diethyl malonate and sodium t-butoxide in the presence of copper(I) chloride, hydrolizing the bis ester to the bis carboxylic acid, and then decarboxylating the resulting 2,4,5-trifluorophenyl malonate to provide 2,4,5-trifluorophenylacetic acid.

Formation of the trifluorophenyl-di(C₁-C₄)alkylmalonate ester is carried out in a suitable solvent. Examples include 1,2-dimethoxyethane (DME), dioxane and the like.

In a preferred embodiment the deprotonating agent used is sodium tert-butoxide, the preferred copper salt used is copper(I) chloride, and the preferred di(C₁-C₄)alkylmalonate is diethylmalonate.

The preferred quantity of deprotonating agent ranges from about 2 to 3 equivalents; preferably about 2.5 equivalents; the preferred amount of copper(I) salt ranges from about 0.25 to 2 equivalents which is a non-stoichiometric amount; the preferred quantity of di(C₁₋₄)alkylmalonate ranges from about 2-3 equivalents, more particularly, about 2.5 equivalents; and the preferred amount of halogenated-trifluorobenzene ranges from about 0.5 to 1 equivalents.

The preferred temperature range following the addition of copper(I) chloride and the halo-trifluorobenzene to the reaction is about 70-95° C.

Conversion of the trifluorphenyl di(C₁₋₄)alkylmalonate to trifluorophenylacetic acid is preferably carried out in aqueous solution.

In another aspect of the invention that is of interest, the de-esterifying agent is preferably NaOH, present in an amount ranging from about 5 to 7 equivalents, more preferably about 6.5 equivalents.

The preferred temperature range during NaOH addition is about 40 to 60° C.; more preferably about 50-55° C.

Following the reaction with NaOH, the aqueous layer is acidified, preferably to about pH 0.5 to 1.5

During de-esterification, the temperature range is maintained at about 65-95° C., preferably about 70-90° C.

In an aspect of the invention that is of particular interest, 1-bromo-2,4,5-trifluorobenzene is converted to the corresponding diethylmalonate by a copper(I) chloride catalyzed reaction with diethylmalonate in a mixture of dioxane or DME and sodium tert-butoxide at about 75-90° C.

In another aspect of the invention that is of particular interest, 2,4,5-trifluorophenyl-diethylmalonate is converted to 2,4,5-trifluorophenylacetic acid using NaOH for de-esterification, and HCl for decarboxylation in water at about 50-90° C.

The following examples are provided for illustrative purposes, and are not intended to be limitations on the disclosed invention.

The starting materials are either commercially available or known in the literature, and some are prepared following literature methods. Purification procedures include e.g., distillation, crystallization and normal or reverse phase chromatography.

### EXAMPLE 1

### 2,4,5-Trifluorophenylactic Acid

CuCl Promoted Malonate Reaction: Step One.

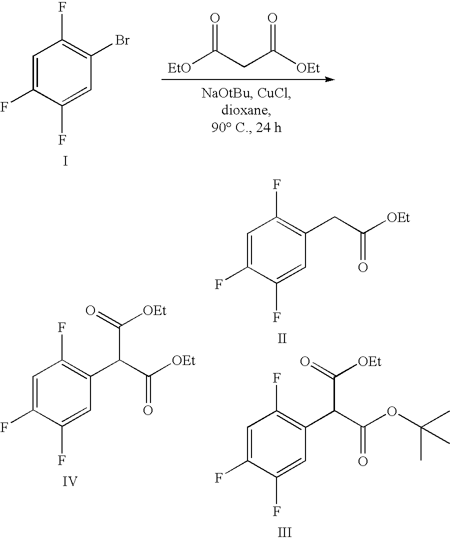

A 3-L mechanically stirred 4-neck reaction vessel was charged with NaOtBu. Anhydrous dioxane (Aldrich anhydrous grade, >0.005% water) (840 mL) was added. This was stirred for 20 min at room temperature. To the resulting slurry was added diethyl malonate (304 mL, 2.0 mol) and the internal temperature increased to 60 to 70° C. The solution was then degassed and purged with nitrogen (N₂) three times. The internal temperature was maintained at ca. 60 to 70° C. CuCl (49 g, 0.50 mol) was quickly added, followed by 1-bromo-2,4,5-trifluorobenzene I (211 g, 1.0 mol). The solution was degassed by pulling a vacuum until reflux was achieved, and then purging with N₂. The mixture was then heated at 90° C. After 24 h, there was >5% I remaining. The reaction was cooled to room temperature and quenched with 2N HCl (1.0 L). This was stirred vigorously for 20 min. The mixture was added to a 200 L separatory funnel, and then diluted with methyl t-butyl ether (MTBE) (840 mL) that was used to wash the Buchi reactor. This was shaken, and the aqueous layer was cut. The organics were washed with 2N HCl (2×1.0 L). The total volume of the aqueous washes was 4.0 L.

Hydrolysis/Decarboxylation: Step Two

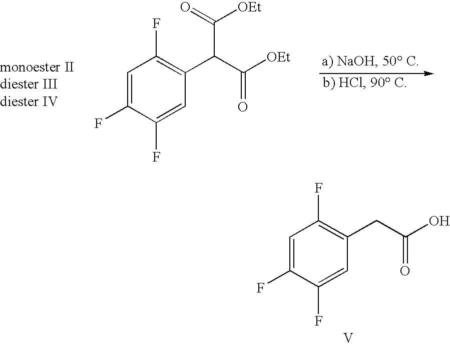

The organics containing a mixture of II, III and IV from the previous malonate coupling were added into a 3-L 4-neck round-bottomed flask. The organics were then evaporated at reduced pressure to about half volume with heating at 40 to 50° C. and water (500 mL) was added. The remaining organics were removed at reduced pressure. To the biphasic mixture was then added NaOH (10 N, 500 mL) so that the temperature rose to 50° C. The mixture was evacuated and the temperature was maintained until the reaction was complete, as determined by liquid chromatographic analysis (a mixture of monoacid and diacid formed, and none of the esters II, III and IV remained), about 30 minutes. The reaction became homogeneous after about 15 min of heating. Upon completion, the reaction was cooled to room temperature, still under reduced pressure. Water (250 mL) was added to bring the total amount of water to 6.0 volumes. MTBE (840 mL) was used to wash the reaction flask, and the combined washing mixture was shaken and allowed to settle over 1 h. The organics were cut and the aqueous layer was added back into the reaction flask. The aqueous layer was acidified with concentrated HCl to pH 1 (ca. 350 mL), and heated to 90° C. for 30 minutes. A bubbler was used to monitor carbon dioxide evolution, which was complete as the mixture was heated between 70 to 80° C. The resulting aqueous mixture was cooled to room temperature and filtered; and the flask was washed with water. The solids were dried on a filter pot overnight, giving 162 g of slightly wet material (95.0 A % pure). Then, half of a 9:1 mixture of n-heptane and IPAC (1.05 L total) was added to the flask, followed by the slightly wet solids, and then the rest of the solvents. This was heated to 90° C. until complete dissolution occurred (at about 90° C.), and then cooled slowly to the crystallization point (ca. 85° C.), and the heating stopped to allow cooling to room temperature. The mixture was further cooled to 0° C., filtered and then washed with cold 9:1 n-heptane/IPAC (200 mL) to produce 2,4,5-trifluorophenyl acetic acid.

### EXAMPLE 2

### 2,4,5-Trifluorophenylacetic Acid

CuCl Promoted Malonate Reaction: Step One

A 1 L, mechanically stirred reaction vessel was charged with NaOtBu and CuCl (0.25 mol). Dry (KF>400) 1,2-dimethoxyethane (400 mL) was added with stirring. To the resulting dark slurry was added diethyl malonate (190 mL, 1.25 mol) so that the internal temperature did not exceed 50° C. The solution was degassed and 1-bromo-2,4,5-trifluorobenzene (105.5 g, 0.50 mol) was added. The mixture was then heated to 75° C. The reaction was stopped after 24 hours (>5% starting material remaining (g/L concentration)). The reaction was cooled to room temperature and quenched into a second vessel containing 3N HCl. This was stirred vigorously for 20 min. The mixture was diluted with MTBE (400 mL) which was used to wash the reaction vessel. This was agitated for 20 minutes, allowed to settle for 20 minutes, and then the aqueous layer was cut. The organics were washed thrice with 3N HCl, stored overnight at 4° C., and then used in the next step.

Hydrolysis/Decarboxylation: Step Two

The solution of II, III and IV in MTBE, approximately 1.0 mol from the coupling step, was added into a 3 L mechanically-stirred reaction vessel with a reflux condenser. Water (670 mL) was added. To the stirring biphasic mixture was then added NaOH (18.9 N, 330 mL, 6.25 mol) and temperature was maintained below 55° C. This temperature was maintained for 1.5 h, until liquid chromatography analysis indicated reaction completion (determined by g/L of IV and II (each <0.5%) in the organic layer). After cooling to RT, n-heptane (400 mL) was added. After agitation for 20 minutes and settling, the organics were cut. The aqueous layer was evacuated and heated to 50° C. to remove the ethanol that was formed in the hydrolysis. The volume was reduced from 1420 mL to 950 mL. Gas chromatography indicated that the ethanol concentration was <0.5 vol %. Water (250 mL) was added. The aqueous layer was warmed to 50° C., then carefully acidified with conc. HCl to pH 1.0 (+/−0.1). Vigorous CO₂ off-gassing was observed upon acid addition, and the temperature rose to about 60° C. over 30 min. The mixture was warmed to 70° C. and maintained for 2 hours. Decarboxylation was monitored by assaying the solid material for the A % ratio of monoacid:diacid (completion at >100:1 A % ratio). The resulting aqueous mixture was then cooled to room temperature. IPAc (800 mL) was added, dissolving the solids completely. After agitation for 20 minutes and settling, the aqueous layer was cut. To the organics was added water (400 mL). The pH of the mixture was ˜1.5, and was adjusted to pH 3.4 with 1N NaOH. The mixture was agitated for 20 minutes, settled and the aqueous layer was cut. The organic layer (930 mL) was stripped to 460 mL and IPAc (440 mL) was added slowly, maintaining a volume of 460 mL to reduce the KF from ˜12,000 to ˜1200 ppm. Then n-heptane (650 mL) was added, and an off-white solid precipitate was formed. The mixture was evacuated and n-heptane (600 mL) was added while maintaining a constant volume (1110 mL). The IPAc/n-heptane ratio was 85:15 (by gas chromatographic analysis of a filtered aliquot). The mixture was heated to 90° C., cooled slowly to 75° C. and maintained at this temperature for 1 h, and cooled slowly to 2° C. The mixture was filtered and the resulting 2,4,5-trifluorphenylacetic acid was washed with IPAc/n-heptane (200 mL, 85:15 ratio), and oven-dried overnight at 40° C., under vacuum (25 mm).

While the invention has been described and illustrated with reference to certain particular embodiments thereof, changes, modifications, substitutions, deletions, or additions of procedures and protocols may be made without departing from the spirit and scope of the invention.

What is claimed is:

1. A process for the preparation of a compound of the formula 1: 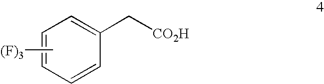
comprising: reacting a compound of the formula 3: 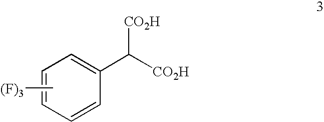
with a decarboxylating agent to produce a compound of formula 4.

2. A process in accordance with claim 1 wherein the decarboxylating agent is an acid selected from the group consisting of hydrochloric, p-toluenesulfonic and sulfuric acids.

3. A process in accordance with claim 2 wherein the decarboxylating agent is hydrochloric acid.

4. A process in accordance with claim 3 wherein the compound of formula 4 is 2,4,5-trifluorophenylacetic acid.

5. A process for the production of a compound of formula 4: 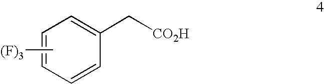
comprising:
a) reacting a compound of formula 1: 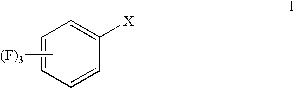
wherein X is a halo group selected from chlorine, bromine and iodine, with a compound of the formula:
ROOC—CH₂—COOR
wherein each R represents a C₁₋₄ alkyl group, in the presence of a deprotonating agent and a copper (I) salt, to produce a compound of formula 2: 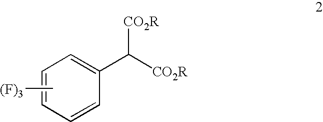
b) reacting compound 2 with a de-esterifying agent to produce a compound of formula 3: 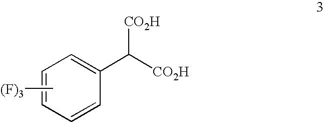
and
c) reacting the compound of formula 3 with a decarboxylating agent to produce a compound of formula 4.

6. A process in accordance with claim 5 wherein each R represents ethyl.

7. A process in accordance with claim 5 wherein the deprotonating agent is sodium tert-butoxide.

8. A process in accordance with claim 5 wherein the reaction is carried out in an aqueous environment.

9. A process in accordance with claim 5 wherein the decarboxylating agent is an acid selected from hydrochloric, p-toluenesulfonic and sulfuric acids.

10. A process in accordance with claim 9 wherein the acid is hydrochloric acid present in an amount sufficient to adjust the pH to about 0.5 to 1.5.

11. A process in accordance with claim 5 wherein the temperature range is about 45 to 95° C.

12. A process in accordance with claim 5 wherein the compound of formula 4 is 1-bromo-2,4,5-trifluorobenzene.

13. A process in accordance with claim 12 wherein the compound of formula 2 is 2,4,5-trifluorophenyl-diethylmalonate.
